(19) United States
(12) Patent Application Publication
(10) Pub. No.: US 2026/0306160 A1
(43) Pub. Date: Oct. 8, 2026

(54) SAFE ADAPTIVE MACHINE LEARNING-BASED CONTROL FOR COLD ATMOSPHERIC PLASMA BIOMEDICAL DEVICES

(71) Applicant: THE REGENTS OF THE UNIVERSITY OF CALIFORNIA, Oakland, CA (US)

(72) Inventors: Ali Mesbah, Danville, CA (US); Kimberly Chan, Berkeley, CA (US); Georgios Makrygiorgos, Berkeley, CA (US)

(21) Appl. No.: 19/474,847

(22) PCT Filed: Apr. 11, 2024
(86) PCT No.: PCT/US2024/024008

§ 371 Date: Oct. 13, 2025

Related U.S. Application Data
(60) Provisional application No. 63/496,110, filed on Apr. 14, 2023.

(51) Int. Cl. A61B 18/02; A61B 18/00

(52) U.S. Cl. CPC A61B 18/02; A61B 2018/00714; A61B 2018/00767

(57) ABSTRACT

A system to control a cold atmospheric plasma device, including a cold atmospheric device, one or more sensors to measure characteristics of one or more of the plasma and a surface at which the cold atmospheric plasma device is directed, and one or more processors configured to execute code to cause the one or more processors to use a deep neural network (DNN) to provide operating parameters for operation of the device and to receive information from the one or more sensors as feedback on the operation.

### STATEMENT REGARDING FEDERALLY SPONSORED RESEARCH AND DEVELOPMENT

This invention was made with Government support under grants 1912772 and 2130734 awarded by the National Science Foundation. The Government has certain rights in this invention.

### TECHNICAL FIELD

This disclosure relates to plasma medicine, more particularly using model predictive controllers, deep learning controllers, and Bayesian optimization methods to control the process.

### BACKGROUND

Biomedical devices can exhibit multivariable, nonlinear, and intrinsically variable dynamics, often with safety-critical constraints. As a result, a growing interest exists in advanced control of biomedical devices using model predictive control (MPC) and learning-based control strategies.

MPC generally involves a control technique in which the calculated control actions strive to minimize a cost function for a dynamic system having operational constraints, over a finite, receding horizon. A receding horizon generally indicates that the horizon changes at each time step or other iteration.

The notion of learning and adaptation, as well as autotuning, of control policies using closed-loop performance data has received increasing attention. Reinforcement learning (RL) undergoes training based upon rewarding desired behaviors and punishing undesired behaviors. In some instances, policy gradient methods have been used as a popular RL approach to guide policy search within continuous control-input spaces, with particular success for MPC policies.

Due to its use of gradient information, some people see policy-gradient RL as a scalable alternative to the increasingly popular Bayesian optimization (BO) strategy for controller auto-tuning. However, it may have a cost in lower data-efficiency, especially when initialized poorly.

A further consideration involves the safety of biomedical systems. Advances in data-driven control and decision-making capabilities have created significant opportunities for autonomy for biomedical devices. The underlying feature of autonomy is a self-driven optimization of what decision to make next. Interactions with humans make safety a fundamental requirement for autonomous systems.

### BRIEF DESCRIPTION OF THE DRAWINGS

FIG. 1 shows a schematic of an embodiment of system for cold atmospheric plasma (CAP) jet.

FIG. 2 shows graphs of hypervolume improvement and an observed Pareto frontier in the context of thermal dose delivery with the CAP jet.

FIG. 3 shows graphs of state and input profiles of closed-loop experiments for iterations of embodiments.

FIG. 4 shows a graph of observed performance measures from an exploration of an embodiment.

FIG. 5 shows an exemplary feasible set and queries made by using safe Bayesian optimization.

FIG. 6 shows a graphical representation of observed closed-loop profiles from safe Bayesian optimization.

### DETAILED DESCRIPTION OF THE EMBODIMENTS

The embodiments here involve a strategy for adaptive deep learning based approximate MPC (model predictive control), towards personalized and point-of-care biomedical plasma applications. Approximate MPC, which hinges on approximating MPC laws via offline computations of the optimal control problem, enables control of biomedical devices at kHz sampling rates. Deep neural network (DNN)-based approximations of MPC laws are especially attractive due to their low memory footprint and versatile embedded implementations on resource-limited, specialized hardware such as field programmable gate arrays (FPGAs).

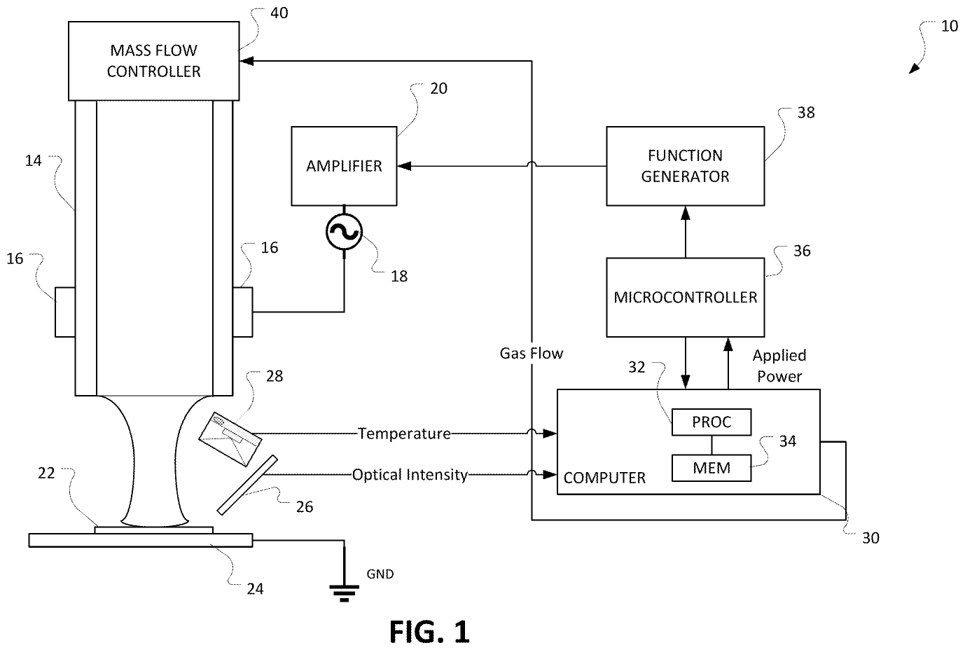

The embodiments involve using a DNN system to approximate an MPC in an environment where the number of trials and data sets have limitations. Controlling a “local” process using a DNN approximation of an MPC has several applications. The system involves one or more processors, as shown in FIG. 1, that receive data from a biomedical device from each “run” or use of the device. The system provides run to run data from several uses to the one or more processors that execute code to perform Bayesian optimization. The processor(s) then adapts a subset of the parameters of the DNN system. The DNN system then provides the settings and otherwise controls the plasma biomedical device in real-time for its next run. Because of the nature of plasma biomedical applications where the number of runs to generate data to provide to the DNN is limited, the nature of the interaction with the DNN is much different than a typical machine learning application that has the advantage of large data sets to train upon.

This type of architecture arose out of a specific solution, that for application of CAP jets to surfaces for wound care and other types of biomedical applications, but the methodology and architecture has wide-ranging applications in cold atmospheric plasma devices.

Cold atmospheric plasmas (CAPs) have recently been found to have promising use in plasma medicine. CAPs, a low temperature (partially) ionized gas, can be generated by applying an electric field to a noble gas, such as argon or helium, whereby the resulting discharge is directed towards a target surface. The synergistic effects of CAPs, including the generation of reactive chemical species and ions, ultraviolet radiation, low-level electric fields, and thermal effects, can induce therapeutic outcomes. As such, portable CAP devices have shown promise for a variety of point-of-care biomedical applications. Thus, there has been a growing interest in advanced control of biomedical CAP devices using model predictive control (MPC), as described in Y. Lyu, L. Lin, E. Gjika, T. Lee, and M. Keidar, “Mathematical modeling and control for cancer treatment with cold atmospheric plasma jet,” Journal of Physics D: Applied Physics, vol. 52, no. 18, p. 185202, 2019 and learning-based control strategies as described in L. Lin and M. Keidar, “Machine learning controlled self-adaptive plasma medicine,” in Proceedings of the IEEE International Conference on Plasma Science, pp. 561-561, 2020.

Two of the main challenges in MPC of CAPs stem from the need to: (i) handle the fast dynamics and, thus, kilohertz (kHz) sampling rates of CAPs as described in A. D. Bonzanini, J. A. Paulson, G. Makrygiorgos, and A. Mesbah, “Fast approximate learning-based multistage nonlinear model predictive control using Gaussian processes and deep neural networks,” Computers & Chemical Engineering, vol. 145, p. 107174, 2021, and (ii) adapt MPC policy parameters to account for variable characteristics of the plasma and target surfaces as described in A. D. Bonzanini, K. Shao, A. Stancampiano, D. B. Graves, and A. Mesbah, “Perspectives on machine learning-assisted plasma medicine: Toward automated plasma treatment,” IEEE Transactions on Radiation and Plasma Medical Sciences, vol. 6, no. 1, pp. 16-32, 2021.

In the area of autotuning control policies, policy gradient methods have been used as a popular reinforcement learning (RL) approach to guide policy search within continuous control-input spaces, with particular success for MPC policies (e.g., M. Zanon and S. Gros, “Safe reinforcement learning using robust MPC,” IEEE Transactions on Automatic Control, vol. 66, no. 8, pp. 3638-3652, 2020). Due to its use of gradient information, policy-gradient RL is touted as a scalable alternative to the increasingly popular Bayesian optimization (BO) strategy for controller auto-tuning (e.g., F. Sorourifar, G. Makrygiorgos, A. Mesbah, and J. A. Paulson, “A data driven automatic tuning method for MPC under uncertainty using constrained Bayesian optimization,” IFAC-PapersOnLine, vol. 54, no. 3, pp. 243-250, 2021.), but at a cost of lower data-efficiency, especially when initialized poorly.

Instead, BO can be a viable alternative for data efficient policy search, especially when performance data and/or interactions with the real environment are limited. BO is a derivative-free, probabilistically principled method for “global” optimization that can handle a mixture of continuous, discrete, and categorical decision variables. For example, A. Marco, F. Berkenkamp, P. Hennig, A. P. Schoellig, A. Krause, S. Schaal, and S. Trimpe, “Virtual vs. real: Trading off simulations and physical experiments in reinforcement learning with Bayesian optimization,” in Proceedings of the IEEE International Conference on Robotics and Automation, pp. 1557-1563, 2017 presented an entropy-search BO approach to use prior information from a “cheap” simulated environment for sample-efficient policy learning on the actual physical system. Moreover, the multi-objective nature of policy search can be directly accommodated in BO when there is a need to discover a set of optimal policies due to multiple conflicting objectives.

For plasma treatment of complex interfaces, it is imperative to adapt control policies to account for the variability among different target surfaces, in addition to the time-varying nature of the plasma and surface characteristics. Moreover, adaptability of the treatment policy is important for personalized plasma medicine, where CAP treatments must be tailored for each individual subject to enhance their therapeutic efficacy without compromising the safety and comfort of patients. However, a key challenge arises from the limited number of treatments/trials that can be performed in a biomedical context, which makes data efficiency a prerequisite for policy adaptation. The CAPJ operation applies to other contexts outside the biomedical context, the biomedical context just provides an example.

To this end, the embodiments present a multi-objective BO (MOBO) strategy for data-efficient and “globally” optimal adaptation of DNN based control policies in a run-to-run manner. MOBO uses probabilistic surrogate models of multiple closed-loop performance measures, such as plasma treatment outcomes, to systematically tradeoff between exploration and exploitation of a subset of DNN parameters. The selection of this subset of parameters is guided by a global sensitivity analysis that quantifies the influence of each network parameter on the performance measures. As such, MOBO yields a data-efficient scheme for performance-oriented adaptation of DNN-based control policies. The embodiments experimentally demonstrate the proposed strategy for adaptive DNN-based approximate MPC of a CAP jet (CAPJ) with prototypical applications in processing of heat-sensitive biomaterials, as an example of a biomedical device with a local control process managed by a DNN system.

One can consider the control problem for a prototypical CAPJ in the context of personalized plasma treatments. One embodiment uses a kHz excited CAPJ in helium (He) that consists of a copper ring electrode wrapped around a quartz tube as described in D. Gidon, D. B. Graves, and A. Mesbah, “Effective dose delivery in atmospheric pressure plasma jets for plasma medicine: A model predictive control approach,” Plasma Sources Science and Technology, vol. 26, no. 8, p. 085005, 2017. A schematic of the CAPJ system 10 is shown in FIG. 1. The computer 30 controls the gas flow to the mass flow controller 40. As the gas flows through the tube, in one embodiment the gas is helium, plasma ignition is achieved by applying a high-frequency alternating current (AC) voltage 18 to the copper electrode 16 under control of the amplifier 20. The plasma is directed out of the tube 14 onto a target substrate, in one embodiment, a grounded, glass-covered 22 metal plate 24 at a distance of 3 mm below the tip of the tube. The applied power P and gas flow rate q are the manipulated inputs from computer 30, which has a processor 32 and a memory 34. The manipulated inputs correspond to the operating parameters resulting from the evaluation of the DNN. The maximum surface temperature T and total optical intensity I of the plasma at the plasma-surface incident point are the measured outputs by IR camera 28 and spectrometer 26, respectively. The IR camera and spectrometer represent different types of sensors to measure characteristics of one or more of the plasma and the surface.

Using data collected from the CAPJ, one can model the system dynamics via a linear time-invariant (LTI) state-space model

$$x ( k + 1 ) = A x ( k ) + B u ( k ) , \qquad ( 1 a )$$

$$y ( k ) = C x ( k ) + D u ( k ) , \qquad ( 1 b )$$

where k is the discrete time step, x∈ [symbol] is the vector of states, u=[P, q]^{T}∈ [symbol] is the vector of manipulated inputs, y=[T, I]^{T}∈ [symbol] is the vector of measured output(s), and A, B, C, D are the state-space matrices identified using subspace identification. The state-space model is defined in terms of deviation variables around a nominal operating condition. Furthermore, one can assume an observable, canonical form of (1), where C=I and D=0. An additional assumption is that the overall system uncertainty is modeled as a stochastic variable w that is added to (1a).

Plasma treatment of complex surfaces relies on quantification of the delivered plasma effects to a surface. The process here describes the accumulation of thermal effects on a target with a metric called cumulative equivalent minutes (CEM), as given by

$$CEM ( k + 1 ) = CEM ( k ) + {K}^{( {T}_{ref} - T ( k ) )} δ t \qquad ( 2 )$$

where K is an exponential base dependent on physical properties of the substrate, T_{ref}=43° C. is the reference temperature, and δt is the sampling time as described in S. A. Sapareto and W. C. Dewey, “Thermal dose determination in cancer therapy,” International Journal of Radiation Oncology Biology Physics, vol. 10, no. 6, pp. 787-800, 1984. This definition of the thermal dose is cumulative in that plasma effects delivered cannot be retracted. With the CEM measure, the augmented system states are x=[T, I, CEM]^{T}. Accordingly, the resulting nonlinear model of the CAPJ for thermal treatment of surfaces takes the form

$$x ( k + 1 ) = f ( x ( k ) , u ( k ) , w ( k ) ) . \qquad ( 3 )$$

The use of CEM provides an example of one of the plasma effects on the surface that can be controlled by the DNN using Bayesian optimization.

The goal of a plasma treatment is to deliver a desired amount of plasma effects as quickly as possible without violating comfort and safety constraints. The embodiments drive to systematically account for inherent uncertainties of CAPJs using a robust MPC formulation, and to this end, uses scenario based MPC (sMPC). sMPC assumes that the system uncertainty is represented by a tree of discrete scenarios, where each branch stemming from a node represents a particular scenario of uncertainty realization. Further, to limit the number of scenarios, a robust horizon Nᵣ is often defined to bound the uncertainty propagation up to a given point. The embodiments here select a “worst-case” formulation of the scenario tree, wherein the scenarios are generated based on the worst-case bounds of the uncertainty. To represent the trajectories generated by S scenarios, the below discussion adopts the notation (x^{j}(i),u^{j}(i)), where the addition of the superscript j indicates the particular scenario

$$j ∈ \{ 1 , … , S \} .$$

As such, the optimal control problem at time step k is formulated as

$${min}_{{x}^{j} , {u}^{j}} {\sum }_{j = 1}^{S} {p}^{j} {V}^{j} ( \cdot ) \qquad ( 4 a )$$

$$s . t . {x}^{j} ( i + 1 ) = f ( {x}^{j} ( i ) , {u}^{i} ( i ) , {w}^{i} ( i ) ) , \qquad ( 4 b )$$

$$( {x}^{j} ( i ) , {u}^{j} ( i ) ) ∈ X \times 𝒰 , \qquad ( 4 c )$$

$${x}^{j} ( 0 ) = x ( k ) , \qquad ( 4 d )$$

$${u}^{j} ( i ) = {u}^{l} ( i ) if {x}^{b ( j )} ( i ) = {x}^{b ( l )} ( i ) , \qquad ( 4 e )$$

$$∀ i ∈ \{ 0 , … , {N}_{p} - 1 \} ,$$

where p^{j} is the probability of a particular scenario, V^{j}(•) is the control cost that can consist of a stage cost over a prediction horizon Nₚ and/or a terminal cost of a particular scenario; w(i) are chosen to be one of three scenarios: zero or proportional to the minimum or maximum error of model identification [α₁wₘᵢₙ, 0, α₂wₘₐₓ], where α₁, α₂∈[0, 1]; (4c) are the state and input constraints; and (4e) enforces a non-anticipativity constraint, which represents the fact that each control input that branches from the same parent node must be equal x^{b(j)}(i) is the parent state of x^{j(i+1)}, and/is another counting variable for a particular scenario l≠j. The solution to (4) defines the sMPC law as

$${π}_{smpc} ( x ( k ) ) = {u}^{*} ( 0 ) \qquad ( 5 )$$

where u*(0) is the optimal first input. Here, the control objective is defined as the terminal cost

$$V ( CEM ( {N}_{p} ) ) = {( {CEM}_{sp} - CEM ( {N}_{p} ) )}^{2} , \qquad ( 6 )$$

where CEMₛₚ denotes the setpoint CEM dose.

Finally, to adapt the policy for personalized CAP treatments, the process focuses on two closed-loop performance measures: (i) the delivery of a desired amount of thermal dose and (ii) the adherence to a comfort/safety constraint. The discussion defines (i) as a CEM tracking cost over the treatment time N

$${ϕ}_{1} = {\sum }_{k = 0}^{N} {( {CEM}_{p} - CEM ( k ) )}^{2} \qquad ( 7 )$$

and (ii) as the sum of the degree of constraint violation in surface temperature over N

$${{ϕ}_{2} = {\sum }_{k = 0}^{N} ( {[ T ( k ) - {T}_{t o l} ]}^{+} ) )}^{2} , \qquad ( 8 )$$

where Tₜₒₗ is the nominal tolerated temperature constraint, and [T(k)−Tₜₒₗ]⁺ is the positive magnitude of constraint violation. These measures are competing since Ttot is often set to a value at or near 43° C., which limits the rate of CEM delivery.

The requirements of embedded control on low-cost, resource-limited hardware for point-of-use CAPJ applications pose a key challenge to online deployment of the sMPC law (5). The challenge arises from the high computational cost of the scenario-tree optimization in (4). To this end, the process uses DNNs to approximate (5). Consider a dataset in the form of

$$τ = {\{ ( {x}_{q} , {π}_{smpc} ( {x}_{q} ) ) \}}_{q = 1}^{{n}_{s}} \qquad ( 9 )$$

representing nₛ state-action (optimal input) pairs as acquired by the offline solution of (4). Let a DNN-based policy be defined as Π: [symbol] A generic feedforward description of a DNN constitutes a nonlinear, input-output mapping, where information is propagated from the input layer to the output layer via L hidden layers that contain H nodes. Given an input z,

$$\prod ( z ; {θ}_{0} , 𝒞 ) = {W}_{L + 1} ∘ ( {σ}_{L} ∘ {W}_{L} ) ∘ … ∘ ( {σ}_{1} ∘ {W}_{1} ) ( z ) , \qquad ( 10 )$$

where

$${θ}_{0} = {\{ {W}_{i} \}}_{i = 1}^{L + 1}$$

are the DNN parameters that are computed via training the DNN using [symbol] ; ∘ denotes the composition operator; and [symbol] denote the hyperparameters. Wᵢ is comprised of the weight matrix and bias between the i-th and (i+1)-th layers and σᵢ is the activation function.

The DNN parameters are generally fit by minimizing a mean-squared-error loss function. Meanwhile, the DNN hyperparameters related to its architecture (e.g., L,

$$H , {\{ {σ}_{i} \}}_{i = 1}^{L + 1} ,$$

as well as the training/fitting options (e.g., learning rate, optimizer solver) must be tuned. Tuning is a crucial step of the DNN policy training since the hyperparameters affect the resource utilization of hardware, meaning the memory required to store the weights/parameters θ₀ and the accuracy of the approximation of (5). The DNN resides on microcontroller 36, programmed by computer 30. The DNN controls the function generator 38 that in turn controls the applied voltage and frequency via the amplifier and electrode. While BO is commonly used to facilitate hyperparameter tuning, the embodiments focus on using BO to adapt DNN parameters. Here, the DNN is trained using closed-loop data generated by solution of sMPC. This way, each step of the closed-loop trajectory is a solution to (5) and represents a suitable situation in which the closed-loop system is likely to operate. Adaptation of DNN-based control policies using data-driven optimization methods such as BO is prone to the curse of dimensionality. To address this, the embodiments utilize a global sensitivity analysis (GSA) with respect to the performance measures φ₁ and φ₂ to decide which candidate parameters θ⊂θ₀ should be prioritized for control policy adaptation.

The control policy adaptation can be cast as a multi-objective (MO) problem characterized by M closed-loop performance measures

$${\{ {ϕ}_{m} \}}_{m = 1}^{M} .$$

The process denotes the closed-loop system uncertainties by d={d(0), . . . , d(N)}, and defines a vector-valued performance measure as h(θ): [symbol] with components hₘ(θ)= [symbol] [φₘ(θ, d)], where θ∈Θ are real-valued decision variables, namely the subset of DNN parameters that are adapted. Then, the MO optimization problem for optimal selection of θ is formulated as

$${min}_{θ ∈ Θ} \{ h ( θ ) s . t . g ( θ ) \geq 0 \} , \qquad ( 11 )$$

where g(θ)≥0 are the constraints. The expectation of each performance measure is approximated in a sample-based fashion as

$${h}_{m} ( θ ) = {𝔼}_{d} [ {ϕ}_{m} ( θ , d ) ] \approx \frac{1}{{N}_{d}} {\sum }_{j = 1}^{{N}_{d}} {ϕ}_{m} ( θ , {d}_{j} ) , \qquad ( 12 )$$

where N_{d} is the number of samples for a given θ. The sample-averaged approximation (12) yields noisy estimates of the performance measures

$${ψ}_{m} ( θ ) = {h}_{m} ( θ ) + {ϵ}^{m} , \qquad ( 13 )$$

where ϵ^{m} represents the noise of the m-th performance measure, and

$$Ψ ( θ ) = {\{ {ψ}_{m} ( θ ) \}}_{m = 1}^{M}$$

denotes the set of observed performance measures for a given θ.

Problem (11) cannot be directly solved in the case of expensive and black-box performance measures. Hence, the general idea of BO is to learn probabilistic surrogate models, typically Gaussian process (GP) models, for the performance measures and select a set of points that jointly optimize the expected value of the current surrogates. This is done by solving a proxy problem where an acquisition function proposes points to query in order to refine the surrogate representing the performance measures. The querying strategy is based on the exploration/exploitation trade-off: the embodiments look to query the measures at points that lie in a neighborhood that can contain the optima while also reducing the prediction uncertainty of the surrogate models. Given newly observed data

$$𝒟 = {\{ ( {θ}_{i} , {Ψ}_{i} ) \}}_{i = 1}^{{N}_{o}} ,$$

each performance measure is updated using Bayesian inference; e.g., for GP surrogates, GP regression is used.

Moreover, in a MO setting, there is not a single best optimizer since the performance measures can be conflicting. Hence, the goal is to discover a set of optimal points, a Pareto frontier comprised of Pareto optimal points. The Pareto frontier is a boundary in the performance measure space in which improving one measure is realized at the expense of degrading the others. Denote a set of Pareto optimal solutions as P. Solutions contained within P are known to be non-dominated by other solutions in the feasible region. For control policy adaptation, Pareto dominance is defined as follows.

Definition 1: Given a set of parameters and their corresponding performance measures {θ,h(θ)}, a solution h(θ_{A}) dominates another solution h(θ_{B}) if hᵢ(θ_{A})≤hᵢ(θ_{B}) ∀i∈{1, . . . , M} and ∃i∈{1, . . . , M} such that hᵢ(θ_{A})<hᵢ(θ_{B}). Pareto dominance is denoted by h(θ_{A})<h(θ_{B}), while a solution h(θ_{A}) is non-dominated if [symbol] θ_{B}∈Θ such that h(θ_{B})<h(θ_{A}). Given Definition 1 and the set of Pareto optimal performance measures, the Pareto frontier is given as

$$P = \{ h ( θ ) s . t . ∄ {θ}^{*} ∈ Θ : h ( {θ}^{*} ) ≺ h ( θ ) \} , \qquad ( 14 )$$

and the set of the Pareto optimal parameters is given as

$$ϑ = \{ θ ∈ Θ s . t . h ( θ ) ∈ P \} . \qquad ( 15 )$$

Establishing a Pareto frontier will enable the selection of optimal control policies, each of which yields optimal performance with varying levels of trade-offs between the performance measures. In MOBO, the search for the Pareto frontier is commonly facilitated by the expected hypervolume improvement (HVI) acquisition function. The expected HVI relies on the definition of an indicator that quantifies the Pareto optimality of the estimated Pareto frontier known as the hypervolume (HV) (S. Daulton, M. Balandat, and E. Bakshy, “Parallel Bayesian optimization of multiple noisy objectives with expected hypervolume improvement,” Advances in Neural Information Processing Systems, vol. 34, pp. 2187-2200, 2021.

Definition 2: The HV is defined with respect to a reference point r∈ [symbol] in the performance measure space. For a finite, estimated Pareto set P, the HV is given as the M-dimensional Lebesgue measure AM of the space dominated by P and bounded by r

$$ℋ𝒱 ( 𝒫 , r ) = {Λ}_{M} ( {⋃}_{i = 1}^{\vert 𝒫 \vert} [ r , {Ψ}_{i} ] ) , \qquad ( 16 )$$

where |P| is the cardinality of P, and [r, Ψᵢ] is the hyperrectangle formed by the points r and Ψᵢ.

The HV acts as a metric to quantify the quality of the Pareto frontier and is affected by the selection of the reference point. Thus, “convergence” to a single HV value means that MOBO has performed enough sampling (based on some prespecified budget) to construct the best possible Pareto frontier, which is not necessarily the true one. Then, the HVI describes the incremental improvement of the HV of P if a new point is added. The HVI of a set of newly observed measures Ψ′ is given by

$$HVI ( {Ψ}^{′} , P , r ) = HV ( {Ψ}^{′} ⋃ P , r ) - HV ( P , r ) . \qquad ( 17 )$$

Hence, the expected HVI acquisition function α_{EHVI} describes the expectation of HVI over the posterior of the performance measures and is given as

$${α}_{EHV I} ( θ ) = 𝔼 [ HVI ( {Ψ}^{′} , P , r ) ] . \qquad ( 18 )$$

Finally, to account for noisy observations of performance measurements, the noisy expected HVI acquisition (NEHVI) is employed; see S. Daulton, M. Balandat, and E. Bakshy, “Parallel Bayesian optimization of multiple noisy objectives with expected hypervolume improvement,” Advances in Neural Information Processing Systems, vol. 34, pp. 2187-2200, 2021. Here, NEHVI is maximized with respect to the DNN parameters θ.

First, the process solves the sMPC problem (4) in closed loop to gather training data for approximating the initial control law (5). In (4), the process sets the prediction horizon Nₚ=5, the robust horizon Nᵣ=2, and the discrete uncertainty scenarios as [0.01wₘᵢₙ, 0, 0.01wₘₐₓ]. The control inputs are constrained by P∈[1.5, 5] W and q∈[1.5, 5] SLM, and the states are constrained by T∈[25, 45]° C. and I∈[20, 80] arb. units. The sMPC was formulated using CasADi and solved with IPOPT. The inventors simulated the true system with a mismatch between the plant and control model and normally distributed measurement noise N(0, (0.1)²). The inventors collected nₛ=5,000 samples of state-to-optimal-input mappings and trained a fully-connected feedforward DNN architecture with L=4, H=7, and ReLU activation functions. The DNN underwent training for 5,000 epochs using PyTorch with the default optimizer settings. The resulting DNN-based policy achieved nearly equivalent performance to the implicit sMPC law. Furthermore, the computation time of the DNN, which depends on the architecture of the DNN, compared to solving (4), on a standard CPU (2.4 GHz quad-core Intel i5 processor) was roughly three orders of magnitude faster (~10⁻⁵ s versus ~10⁻² s).

The process considers the treatment of a subject with characteristics that differ from the mean population values, and the goal is to adapt the initial policy designed for the population mean to cater to the individual subject. Note that the closed-loop performance measures are parameterized by subject-specific characteristics, namely K in the CEM setpoint tracking cost (7) and Tₜₒₗ in the comfort constraint cost (8). Here, one can examine the case in which the parameters of the population mean are Kₚₒₚ=0.5 and T_{tol,pop}=45° C., while the characteristics of the individual subject are K_{indiv}=0.55 and T_{tol,indiv}=44.5° C.

First, one must examine the sensitivity of the DNN-based policy to perturbations in different subsets of its parameters. Knowing that the desire for personalized treatments is to minimize the number of trial-and-error treatments, a subset of DNN parameters were adapted due to its high dimensionality (n_{θ}_{₀}=212). Common practice is to freeze the DNN and adapt the last layer and/or append a new layer to the network to adapt.

To evaluate this practice, the sensitivity of the closed-loop performance measures to the parameters of the first and last layers of the DNN-based policy was examined. To perform a GSA, the process used the sensitivity analysis tools by UQLab [33]. The embodiments used a moment independent method (i.e., Borgonovo indices) to analyze the global sensitivity of the selected DNN parameters to the closed-loop performance measures. The inventors generated 10,000 samples of the 44 parameters encapsulated by the first and last layers of the 4-layer, 7-node DNN. Samples were selected from geometrically-bounded values from the initial policy parameters. For each sample, the process ran triplicate closed-loop simulations using the DNN-based policy and evaluated the mean plus and minus standard error values of the observed closed-loop measures. Table I lists the results of the GSA.

TABLE I
SENSITIVITY VALUES (MEAN ± STANDARD
ERROR) OF THE CLOSED-LOOP MEASURES
TO VARIOUS PARAMETERS OF THE POLICY

| | First Layer | Last Layer |
|---|---|---|
| Thermal Dose Delivery (φ₁) | 0.025 ± 3.2e−4 | 0.026 ± 8.3e−4 |
| Temperature Constraint (φ₂) | 0.036 ± 4.7e−4 | 0.038 ± 1.1e−3 |

In general, the dose delivery measure (7) is less sensitive to changes in the parameters compared to the temperature constraint measure (8). Overall, both of the measures are equally sensitive to all of the parameters selected for GSA. Despite this, the parameters of the last layer have slightly higher influence with fewer number of parameters. Hence, the embodiments selected the last layer of the DNN based policy as the subset of parameters to modify in the policy search procedure (i.e., θ=W_{L+1} such that n_{θ}=16).

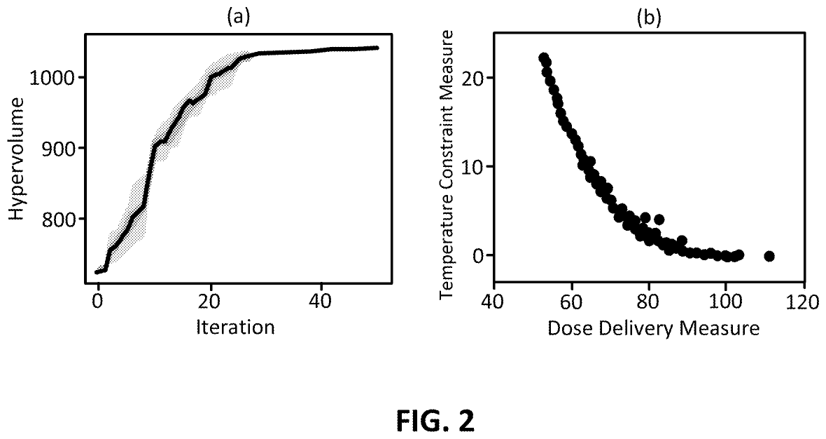

As a global optimization method, MOBO provides a means to systematically explore and detect trade-offs between competing performance measures. FIG. 2 shows the results of 5 replicates of 50 iterations of MOBO on a simulated CAPJ, where one iteration of MOBO is comprised of N_{d}=3 replicates. To implement MOBO, the process used Ax. Ax interfaces with BoTorch to perform BO, and BoTorch interfaces with GPyTorch for the surrogate modeling with GPs. These tools were primarily used with their default settings, using the Matérn 5/2 kernel for GPs and the noisy EHVI acquisition function.

The HV profile in FIG. 2(a) shows the “convergence” of MOBO. The reduced improvement in the HV after more than 30 iterations indicates that MOBO has achieved some optimal representation of the Pareto frontier depicted in FIG. 2(b). While it took 20 or more iterations to achieve this Pareto frontier, the first few iterations of MOBO can drastically improve upon the initial policy. The steep increase in HV suggests that the initial policy parameterization is suboptimal, and a new Pareto optimal point can be found in the first few iterations even when starting with a suboptimal solution.

For the experimental demonstrations of the proposed approach on the CAPJ depicted in FIG. 1, the sMPC had Nₚ=Nᵣ=2, and the input bounds were adjusted to P∈[1.5, 3.5] W and q∈[3.5, 7.5] SLM. Then, 11 closed-loop experiments resulting in nₛ=1,378 samples were performed to gather training data for the DNN approximation. The DNN was trained with the same structure and procedure as described for the simulation studies and achieved similar closed-loop performance to implicit sMPC. MOBO was performed for 15 total iterations due to a limited budget of 45 treatments.

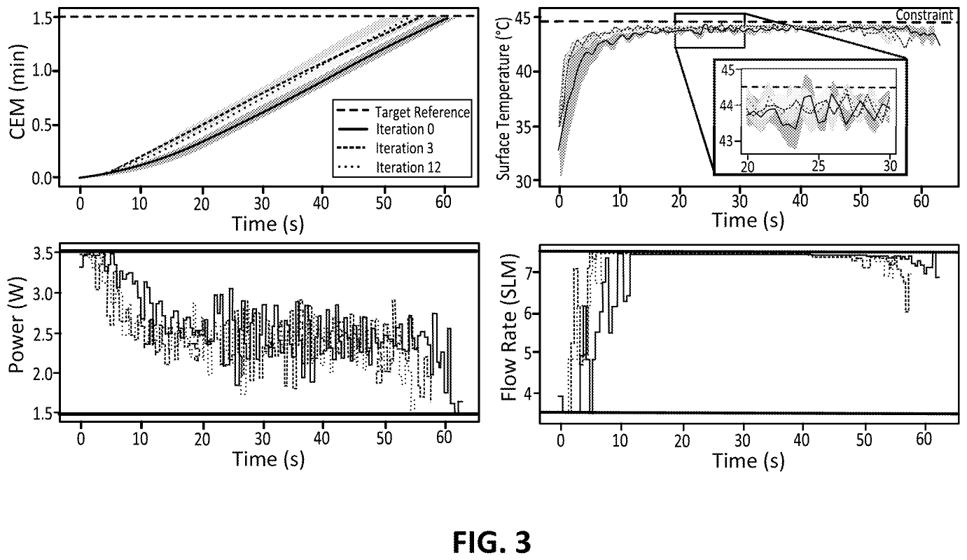

FIG. 3 shows the state and input profiles of 3 particular iterations of MOBO. Within each iteration, the inventors performed N_{d}=3 replicate real-time experiments to account for the intrinsic variability of the system. The CEM profiles are plotted with min-max bounds of the three replicates represented by the shaded region, while the solid line represents the median value of the triplicate runs. The temperature profiles represent the mean value (solid lines) plus and minus two standard errors (shaded region) of the triplicate experiments. Both input profiles are plotted with the mean value from the triplicate experiments. In FIG. 3, the profiles shown are determined to be a few of the “best” treatment options encountered through the process of MOBO.

In this case, the “best” is described in one of two ways: (i) if there was insufficient data to establish a clear Pareto frontier, then the best treatment was considered the initial policy, and (ii) once an estimated Pareto optimal point was found, the best treatment used the policy that produces the lowest constraint violation. This sequence of treatment protocols follows a “safe” treatment intuition. As in for (i), the initial treatment is deemed safe for the general population and is considered “best” for the time being. In the case of (ii), once a Pareto frontier is established, the treatment may then be switched to a more optimal one at the cost of minor temperature violations. Note that establishing some trade-off between the different performance measures via an estimated Pareto frontier allows for the personalization of plasma treatments.

From FIG. 3, the first “best” profile is the initial profile (in blue); a new “best” is encountered after Iteration 3 (in dashed orange). The dashed orange profile represents a new parameterization of the policy that outperforms the initial blue policy, as it achieves the CEM faster (reducing the median treatment time by roughly 8 s or 13%), with slight constraint violations. After more iterations of MOBO, a new policy in dotted green is found at Iteration 12. In this case, the CEM delivery on average is similar to the orange policy (reducing the median treatment time from the initial policy by 10 s or 16%), while maintaining a lower constraint cost.

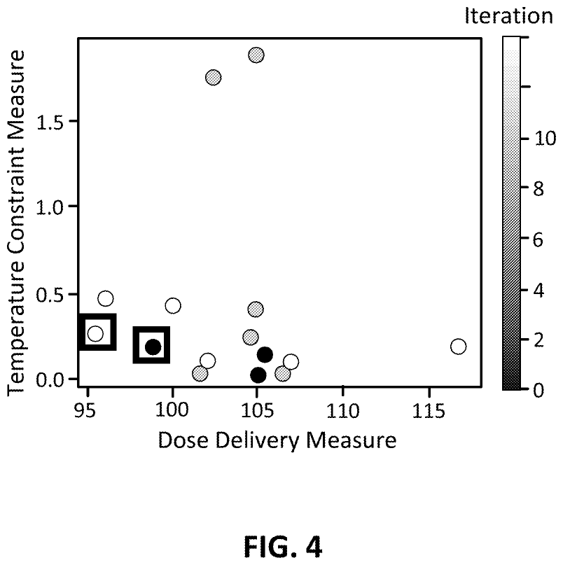

Furthermore, in FIG. 3, the flow rate of He tends to become saturated during treatment. In general, higher He flow rates are characteristic of lower temperatures. Thus, because of the temperature tolerance specification, the operation of the CAPJ necessitates higher flow rate to remain within the region of desirable operating temperatures. The locations of these “best” points in the performance measure space are shown by the red boxes in FIG. 4. With few iterations, the Pareto frontier cannot be visually established, but several points may still be identified as Pareto optimal by their proximity to the minimization of both performance measures. FIG. 4 is consistent with the profiles in FIG. 3 in that Pareto optimal points are found at Iterations 3 and 12 (as indicated by the red boxes). As such, a strategy has been established that can tradeoff between multiple performance measures in order to tailor the treatment to individual subjects.

One constraint on the system that exists in biomedical devices that may not exist in other situations involves the safety of the patients. The discussion now turns to considering the problem of Bayesian optimization (BO) for systems with safety-critical constraints. Recent work has shown that a theoretically consistent way to account for constraints in BO, without losing theoretical global optimality guarantees, is to relax the constraint functions such that the feasible region has a high probability of containing the global solution. However, by construction, these approaches are unable to ensure safe/feasible operation at every query, which is unacceptable in safety-critical applications.

Alternatively, safe BO methods force the query points to remain in the interior of a partially revealed safety region, which may result in unacceptable (and unquantified) performance losses. The performance of safe BO is demonstrated for safe control of a cold atmospheric plasma jet towards personalized plasma medicine (https://ieeexplore.ieee.org/abstract/document/10384190).

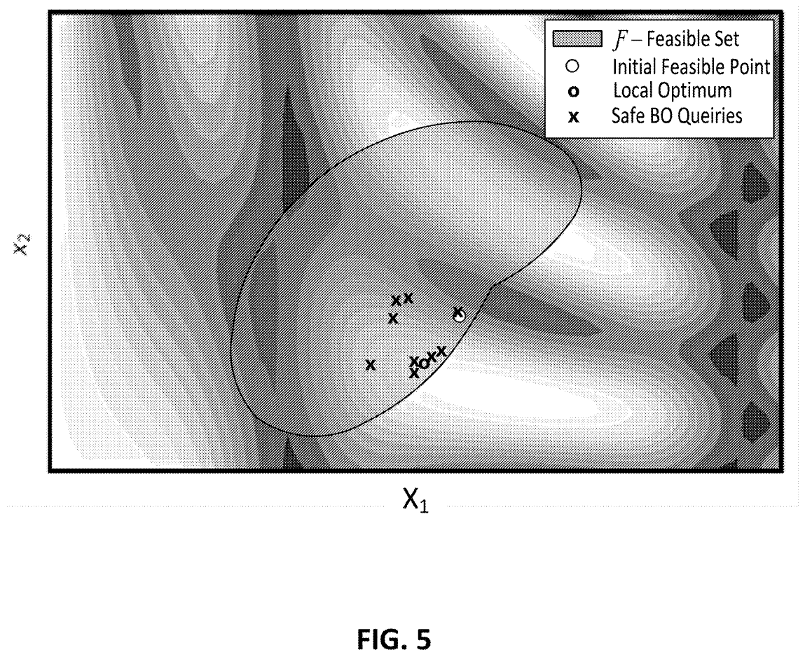

As in the discussion above, while the example of the CAPJ serves as an example and a situation to demonstrate the principles of the embodiments of MOBO, this discussion also applies to any cold atmospheric plasma biomedical or other environments that have to take into account human safety. Safe BO restricts the search space to the known estimated feasible region. The constraints in (11) above, from a safety-critical perspective, must be satisfied at all times. BO provides the framework in which the embodiment uses to modify the control of plasma medical devices. It has the flexibility to be adjusted based upon the application and/or user preferences. This allows it to be applied to plasma medical devices. FIG. 5 depicts how BO restricts the search space to the known estimated feasible region.

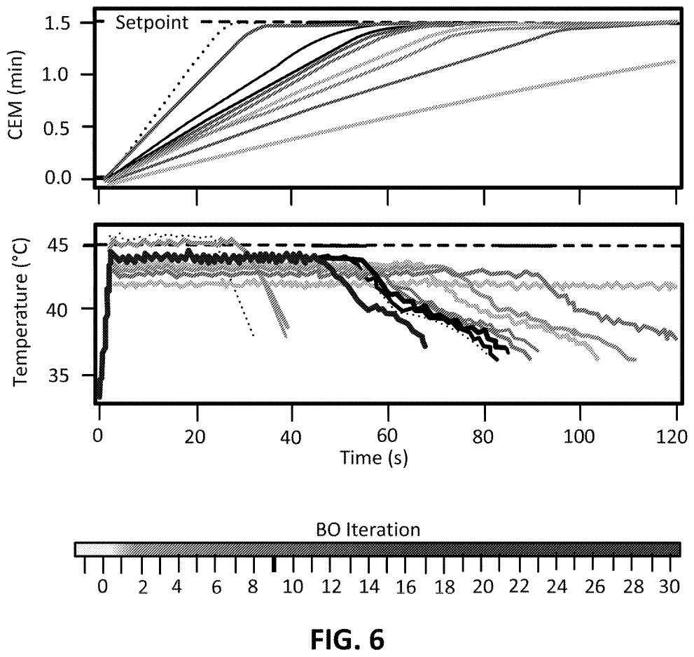

FIG. 6 shows a graphical representation of the observed closed-loop profiles for safe Bayesian optimization. This was generated using model predictive controller, and did not include a neural network controller, but the principle of modifying parameters of the controller as discussed above is the same. The top figure represents the evolution of CEM over a treatment period of 120 s. The bottom figure represents the evolution of temperatures over the same treatment period. The colors/gradients of the profile indicate the evolution of the profiles over 30 iterations of BO. The first two profiles in dotted pink indicate the initial data provided to the BO. FIG. 6 demonstrates that safe Bayesian optimization does suggest queries that are safe within a reasonable probability.

The embodiments demonstrate safe Bayesian optimization for an example application for personalized plasma medicine, which is an emerging field of study involving the use of cold atmospheric plasmas (CAPs) for a variety of medical treatments as described in M. Laroussi, S. Bekeschus, M. Keidar, A. Bogaerts, A. Fridman, X. Lu, K. Ostrikov, M. Hori, K. Stapelmann, V. Miller, et al., “Low temperature plasma for biology, hygiene, and medicine: Perspective and roadmap,” IEEE Transactions on Radiation and Plasma Medical Sciences, vol. 6, no. 2, pp. 127-157, 2022. Tailoring the plasma effects applied to a particular surface/subject is key to ensuring the efficacy of plasma treatments as described in M. A. Hamburg and F. S. Collins, “The path to personalized medicine,” New England Journal of Medicine, vol. 363, no. 4, pp. 301-304, 2010. However, the underlying mechanisms of plasma-surface interactions can only be quantified for a population as described in J.-M. Pouvesle and E. Robert, “Multimodal action of atmospheric pressure plasma jets for biological applications,” in ISPB 2017, 2017, and A. D. Bonzanini, K. Shao, A. Stancampiano, D. B. Graves, and A. Mesbah, “Perspectives on machine learning-assisted plasma medicine: Toward automated plasma treatment,” IEEE Transactions on Radiation and Plasma Medical Sciences, vol. 6, no. 1, pp. 16-32, 2021. Therefore, iterative improvements in automated treatments using BO will enable the personalization of CAP treatments, wherein ensuring (patient) safety is of the utmost importance.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the embodiments.

We claim:

1. A system to control a cold atmospheric plasma device, comprising:
a cold atmospheric biomedical device;
one or more sensors to measure characteristics of one or more of the plasma and a surface at which the cold atmospheric plasma device is pointed; and
one or more processors configured to execute code to cause the one or more processors to use a deep neural network (DNN) to provide operating parameters for operation of the cold atmospheric plasma device and to receive information from the one or more sensors as feedback on the operation.

2. The system as claimed in claim 1, the one or more processors are further configured to collect results from operation of the cold atmospheric device in a run-to-run manner.

3. The system as claimed in claim 2, wherein the one or more processors are further configured to execute code to cause the one or more processors to apply Bayesian optimization to run-to-run data gathered from operation of the cold atmospheric plasma device to produce control parameters for the DNN to implement a control policy.

4. The system a claimed in claim 3, wherein the Bayesian optimization comprises one of safe Bayesian optimization, multi-objective Bayesian optimization, or safe multi-objective Bayesian optimization.

5. The system as claimed in claim 1, wherein the one or more processors are selected to be resource-limited hardware for embedded control.

6. The system as claimed in claim 1, wherein the one or more processors are further configured to ensure that the outputs of the deep neural network fall within a constrained range of values that is larger than an initial range of values thought to be safe.

7. The system as claimed in claim 1, wherein the DNN receives as inputs temperature and optical intensity of the cold atmospheric plasma jet.

8. The system as claimed in claim 1, wherein the DNN has an output of one or more of applied power, applied voltage, frequency, and gas flow rate.

9. A method of controlling a cold atmospheric plasma device, comprising:
identifying operating parameters for operation of the cold atmospheric plasma device that have the most influence on a positive result of the operation;
providing data from each operation of the cold atmospheric plasma device to a deep neural network (DNN) to allow the deep neural network to provide optimum values for the identified operating parameters;
operating the cold atmospheric plasma device using those parameters; and
providing the data from the operating back to the deep neural network.

10. The method as claimed in claim 9, wherein the data comprises optical intensity and temperature.

11. The method as claimed in claim 9, wherein the operating parameter comprises one or more of applied power, applied voltage, frequency, and gas flow rate.

12. The method as claimed in claim 9, wherein the system applies Bayesian optimization to the data from the operation of the cold atmospheric plasma device to adapt control parameters of the DNN to implement a control policy.

13. The method as claimed in claim 12, wherein the Bayesian optimization comprises one of safe Bayesian optimization or multi-objective Bayesian optimization.
